(19) United States
(12) Patent Application Publication
(10) Pub. No.: US 2011/0105782 A1
(43) Pub. Date: May 5, 2011

(54) Process for the preparation of carbamates of primary and secondary polyamines

(76) Inventor: Maurizio Masi, Milano (IT)

(21) Appl. No.: 11/629,617

(22) PCT Filed: Apr. 18, 2005
(86) PCT No.: PCT/IB05/01091

§ 371 Date: Nov. 1, 2007

(30) Foreign Application Priority Data
Jun. 18, 2004 (IT) MI2004A001229

(51) Int. Cl. C07C 269/04

(52) U.S. Cl. 560/33; 560/114; 560/232

(57) ABSTRACT

A process is described for the preparation of carbamates of primary and/or secondary polyamines, which process comprises the steps of vaporising a primary and/or secondary polyamine and reacting said vaporised primary and/or secondary polyamine with gaseous carbon dioxide. In particular, the process relates to the production of the carbamates of hexamethylenediamine, 1,4-diaminocyclohexane and 3-aminomethyl-3,5,5-trimethylcyclohexylamine.

The present invention relates to a process for the preparation of carbamates of primary and secondary polyamines. In particular, it relates to the production of the carbamates of hexamethylenediamine, 1,4-diaminocyclohexane and of 3-aminomethyl-3,5,5-trimethylcyclohexylamine.

Polyamine carbamates, in particular the diamine carbamates, are known as crosslinking agents for elastomers. They are in particular used as crosslinking agents for fluoroelastomers (FKM), polyacrylic elastomers (ACM), ethylene-acrylic elastomers (EAM) and epichlorohydrin elastomers (ECO), because, at vulcanisation temperatures, they are converted into the polyamines, which are the actual crosslinking agents. The widespread use of carbamates in this field is due to the fact that they are solid derivatives of polyamines, which in contrast are in general liquid, polluting and corrosive. Furthermore, the decomposition reaction of the carbamate results in the formation of carbon dioxide, which is capable of forming finely divided foams.

Polyamine carbamates are conventionally produced by reacting a polyamine in solution with gaseous or dissolved carbon dioxide, as described in patents U.S. Pat. No. 3,029,227, US 1972-264,830 (GB 1,374,645) and FR 1,440,467. For example, hexamethylenediamine carbamate has been prepared by reaction between hexamethylenediamine and carbon dioxide in chlorobenzene as solvent, and ethylenediamine carbamate has been produced by reaction of ethylenediamine with carbon dioxide in the presence of toluene or methanol.

Alternatively, according to the teaching of U.S. Pat. No. 4,102,801, polyamine carbamates have in the past been produced by absorbing the polyamine onto a particulate carrier and then reacting the polyamine with carbon dioxide, in the absence of solvent.

Since polyamines exhibit elevated reactivity with carbon dioxide and, on the basis of the observation that hexamethylenediamine in the solid state reacted at low temperatures with the carbon dioxide present in the atmosphere (Leon Segal in Applied Spectroscopy Vol. 17, No. 1, 1963 on pages 21-22), the attempt has been made to prepare polyamine carbamates by a solid state reaction of the polyamine with the carbon dioxide present in the atmosphere. However, the course of the reaction was greatly obstructed by the resistance to the mass transport of carbon dioxide in the condensed phase and, for this reason, this way of performing the reaction has not been converted into an efficient method for the industrial preparation of polyamine carbamates.

A recent synthesis of diamine carbamate salts has been described in international application, publication no. WO9729083. The synthesis method involves reacting a sprayed liquid diamine with gaseous carbon dioxide, optionally in the presence of nitrogen as carrier gas. This method has the advantage of producing dry salts without the necessity of evaporating the solvent, but makes it difficult to control the size distribution of the particles of carbamate obtained. Particle size distribution is in fact an important property in carbamates, in particular if they are to be used as crosslinking agents. The grain size distribution of the carbamate used is in fact capable of influencing the mechanical properties of the polymer, since the decomposition kinetics of the carbamate particles are faster for smaller particles.

Polyamine carbamates are accordingly generally sold as fine powders, the particle diameter of which is generally less than 20 microns, with a mean value of approx. 5-6 microns.

According to the teaching of international application WO9729083, the liquid diamine is sprayed into the gaseous carbon dioxide by making use of liquid atomisers which produce droplets of polyamine generally of a size of 50 microns. The process described in the international application is thus not capable of guaranteeing the required liquid droplet sizes, and thus, once the carbamate is obtained, it must be subjected to a powder micronisation phase in order to obtain the desired particle size distribution.

The object of the present invention is thus to provide a method which does not use solvents and so makes it possible to dispense with the drying phase and which simultaneously avoids the micronisation phase of the carbamate obtained.

A further object of the invention is to allow the use of small reaction volumes and a desired particle size distribution to be obtained.

The above-stated objects have been achieved by the process for the preparation of polyamine carbamates according to claim 1.

Further features and advantages of the present invention will emerge from the following detailed description provided with reference to some examples of embodiment, which are stated by way of non-limiting examples.

The invention accordingly relates to a process for the preparation of carbamates of primary and/or secondary polyamines comprising the steps of a) vaporising a primary and/or secondary polyamine and b) reacting said polyamine in vapour phase with gaseous carbon dioxide.

Suitable polyamines include primary and secondary aliphatic, cycloaliphatic and aromatic diamines, preferably having a number of carbon atoms in the range from 2 to 14.

Representative primary diamines, which may be used in the process of the present invention, include, but are not limited to, 1,3-diaminopropane, 1,6-diaminohexane (hexamethylenediamine), 1,2-diaminoethane, 1,4-diaminobutane, 1,5-diaminopentane, 1,10-diaminodecane, 1,12-diaminododecane, 1,4-diaminocyclohexane, 4,4′-methylenebis(cyclohexylamine), 1,4-phenylenediamine, 1,3-phenylenediamine, 1,4-diaminocyclohexane and 3-aminomethyl-3,5,5-trimethylcyclohexylamine.

The primary diamine is preferably selected from the group consisting of 1,6-diaminohexane (hexamethylenediamine), 1,12-diaminododecane, 4,4′-methylenebis(cyclohexylamine), 1,4-diaminocyclohexane and 3-aminomethyl-3,5,5-trimethylcyclohexylamine, and still more preferably it is 1,6-diaminohexane (hexamethylenediamine), 4,4′-methylenebis(cyclohexylamine) and 1,4-diaminocyclohexane.

Representative secondary diamines, which may be used in the process of the present invention, include, but are not limited to, pyrazolidine (1,2-dimethyldiaziridine), piperazine (1,4-diazacyclohexane), 1,4-diazacycloheptane, 2-methylpiperazine or 2,6-dimethylpiperazine.

If the polyamine used is solid at ambient temperature, it must first be liquefied and then subjected to the vaporisation step a) of the process according to the invention.

The process is described here by way of example in relation to the synthesis of carbamates of primary polyamines, but may readily also be applied to the synthesis of carbamates of secondary or mixed (primary and secondary) polyamines by the person skilled in organic synthesis.

The process according to the invention provides a step a) of vaporising the primary, secondary or mixed polyamine. The vaporisation step may be performed in accordance with any method known to the person skilled in the art by using a suitable vaporisation apparatus. For example, the polyamine may be vaporised either by reducing the operating pressure in a flash chamber or by bubbling a carrier gas into the liquid polyamine. As indicated above, if the polyamine used is in the solid state at ambient temperature, it must be subjected to a melting step which transforms it into the liquid phase, before it is introduced into the vaporisation apparatus. A melting chamber at a suitable temperature is preferably used.

The bubbling apparatus or the flash chamber must be maintained at a temperature higher than the melting temperature of the starting material, the polyamine, in order to ensure that it remains in the liquid state and to facilitate handling and feeding operations. The flow rate of the carrier into the bubbling apparatus or the pressure in the flash chamber must be selected as a function of the amount of polyamine to be vaporised. The carrier gas is preferably an inert gas and still more preferably it is selected from the group consisting of nitrogen, air and nitrogen-enriched air.

The partial pressure in the polyamine vapour phase depends on the temperature selected in the vaporisation apparatus, this temperature being determined by the vapour pressure of the polyamine itself according to the thermodynamics of the liquid-vapour equilibrium. The vapour pressures of the diamines selected for performance of the process according to the invention may be found, for example, in the database of the American Institute of Chemical Engineers (American Institute of Chemical Engineers DIPPR) or the database of the American National Institute of Standards & Technology (NIST).

If a bubbling apparatus is used to vaporise the polyamine, a carrier gas is preferably injected into the liquid polyamine in such a manner that the bubbling height is sufficient to ensure that conditions of thermodynamic equilibrium are achieved.

If a flash chamber is used to vaporise the polyamine, once the temperature of the chamber has been set, the pressure must be selected as a function of the amount to be vaporised and the level of vacuum subsequently available for the reaction chamber. The vaporisation process may also be facilitated by optionally supplying an inert carrier gas to the flash chamber.

In both cases, the carrier gas is preferably nitrogen. According to this preferred embodiment of the invention, the nitrogen which leaves the reactor after the reaction step b) may be recirculated upstream of the vaporisation apparatus, eliminating therefrom any carbon dioxide, which is optionally present in excess, by means of a suitable trap. The trap may be obtained by bubbling the carbon dioxide contaminated nitrogen gas into a sacrificial polyamine melt or into an alkaline solution. The presence of the trap is necessary to avoid the formation of the carbamate salt in the vaporisation apparatus, where the recirculated nitrogen is reintroduced as carrier gas.

According to the invention, the most preferred polyamine is hexamethylenediamine. The melting point of prepared, commercially available hexamethylenediamine is 39-41° C. and this substance must accordingly be heated to above this temperature in order to be melted prior to vaporisation. As stated above, for all the primary and/or secondary starting polyamines, the partial pressure in the vapour phase of the polyamine thus depends on the selected temperature of the vaporisation apparatus.

Step b) of the process according to the invention is then the reaction between the polyamine in the vapour phase and carbon dioxide. The vaporised primary and/or secondary polyamine from step a) is injected into the carbon dioxide and the reaction between these two reactants proceeds vigorously, immediately and completely.

Without wishing to be bound by any particular theory, it is believed that the fact that both the reactants are in the gas phase, as provided by the present invention, ensures immediate and intimate contact and the rapid reaction of the polyamine with the carbon dioxide with complete conversion into the desired carbamate. Indeed, the rapid reaction of the polyamine and the carbon dioxide means that particle nucleation occurs virtually instantaneously, these particles growing rapidly by coalescing with one another and by direct deposition of material due to the chemical reaction.

Preferably in step b), the polyamine, the carbon dioxide, or both may be mixed with a propellant gas to ensure still more intimate and immediate contact between the two reactants. Still more preferably, the propellant gas is an inert gas selected from the group consisting of air, nitrogen and nitrogen-enriched air and is supplied to the reactor independently of the polyamine and the carbon dioxide.

According to the described embodiment of the invention, the vaporised polyamine, in the optional presence of the carrier gas, is then injected into the reactor through a suitable nozzle. The injection nozzle may be designed in accordance with the theory of reactors for aerosol systems, as for example described in T. T. Kodas & M. Hampden-Smith, Aerosol Processing of Materials, Wiley 1999 on pages 293 et seq. in order to obtain the desired particle size distribution. Nozzles with multiple orifices and multiple injection points may provide a still more preferred desired particle size distribution of the polyamine carbamate salt.

The carbon dioxide is preferably introduced into the reactor at ambient temperature through a nozzle path or nozzle section which is separate from that for the injection of the diamine so as to avoid the formation of solid carbamate at the nozzle. Alternatively, the carbon dioxide may be introduced into the reactor after preheating which adjusts it to the desired reaction temperature. Specifically, said reaction temperature is at least that necessary to maintain the polyamine in the vapour phase, while simultaneously avoiding the decomposition of the carbamate obtained.

The synthesis reactor may be operated in either laminar or turbulent fluid dynamic regime. The choice depends on the desired particle size distribution.

With the aim of achieving complete conversion of the polyamine and obtaining a complete reaction, the carbon dioxide must be present in the reaction zone in an amount which is at least stoichiometrically equivalent to the amount of polyamine. The carbon dioxide is preferably present in the reaction mixture in stoichiometric excess.

The propellant gas optionally used for the carbamate formation reaction may simply be introduced into a jet section which is suitable for controlling mixing of the two reactants and the subsequent reaction thereof. The purpose of this separate stream is to prevent the reaction from back-proceeding to the nozzle and to prevent the formation of solid deposits, due to premature pre-contact between the two reactants, from blocking the nozzle itself.

Since carbamates are unstable at elevated temperature, the reaction temperature should be below the decomposition temperature of the selected polyamine carbamate. However, decomposition of the carbamate in the reactor may be inhibited by the overpressure of the carbon dioxide which is present in the reactor in stoichiometric excess.

According to the embodiment of the invention described above, the vaporisation apparatus and the diamine reaction apparatus are separate. This has the advantage of allowing a different operating temperature in the two sections and so maintaining control over each of the following aspects: vaporisation of the polyamine, the decomposition thereof and the decomposition of the final carbamate salt.

However, according to an alternative embodiment of the invention, a single apparatus may be provided which nevertheless allows primary vaporisation of the diamine and the reaction thereof in the vapour state with subsequently introduced gaseous carbon dioxide.

The synthesis reactor according to the invention intended for the performance of step b) may operate under continuous conditions or alternatively under semi-continuous conditions, namely with discontinuous discharge of the carbamate powder.

The process of the present invention accordingly avoids the problems of handling solvent before and during the reaction and simultaneously avoids the performance of micronisation processes or the provision of a cryogenic grinding phase.

The vapour phase synthesis according to the invention thus makes it possible to obtain the desired polyamine carbamate in suitable particle sizes, specifically because of the high degree of control of particle size distribution which can be achieved with aerosol processes performed in the vapour phase.

All the following Examples were performed using an aerosol reactor, in which, in order to distribute the gas, the polyamine and the carbon dioxide in a cocurrent configuration, a concentric nozzle with two rings was used to avoid premixing of the two reactants prior to the reaction chamber. The reactor outlet was equipped with a porous porcelain filter to collect the solid formed by the reaction. The cocurrent plant configuration used here is not restrictive, in that it is also possible to inject the reactant gases into the reactor countercurrently, namely by injecting the gases by means of two nozzles which are in opposition one another.

### EXAMPLE 1

### Preparation of Hexamethylenediamine Carbamate

Hexamethylenediamine, 99.5% purity, was charged into a saturator (or bubbling apparatus) and electrically heated to 100° C. while maintaining at a pressure of slightly above one atmosphere in the apparatus. Nitrogen gas was then supplied to the bubbling apparatus at a flow rate of 100 standard litres per minute, bringing about the vaporisation of 17.8 g/min of hexamethylenediamine. The resultant stream of gas was then introduced into an aerosol reactor, maintained at the same temperature as the bubbling apparatus, and 4 standard litres per minute of carbon dioxide were simultaneously supplied. After 1 hour's operation, 1.47 kg of solid powder had been collected. It was demonstrated that the solid powder collected was hexamethylenediamine carbamate by means of differential scanning calorimeter analysis (DSC) with a decomposition point of 154° C. The powders obtained and collected proved to be a fine dispersion with particle size of less than 3 microns.

### EXAMPLE 2

### Preparation of Hexamethylenediamine Carbamate

Hexamethylenediamine, 99.5% purity, was charged into a saturator (or bubbling apparatus) and electrically heated to 115° C. while maintaining at a pressure of approx. 50 mm Hg by means of a vacuum pump. Said vacuum pump, connected in series to the aerosol reactor, exhibited a volumetric flow rate which enabled the vaporisation of 13.3 g/min of hexamethylenediamine. The resultant stream of vapour was then supplied to an aerosol reactor, maintained at the same temperature as the bubbling apparatus in order to avoid condensations, and 3 standard litres per minute of carbon dioxide were simultaneously supplied. After 1 hour's operation, 1.10 kg of solid powder had been collected. It was demonstrated that the solid powder collected was hexamethylenediamine carbamate by means of differential scanning calorimeter analysis (DSC) with a decomposition point of 154° C. The powders obtained and collected proved to be a fine dispersion with particle size of less than 4 microns.

### EXAMPLES 3-8

The procedure described in Example 1 was repeated, but changing the reference polyamine. In the case of solid polyamines, the polyamines were introduced into the bubbler maintained at a temperature capable of keeping them in the liquid state. The carrier gas used was nitrogen. Examples 3-8 were then performed, the operating conditions used and the results obtained being summarised in Table 1.

TABLE 1
Examples 3-8

| | | | | Flow rate of | | | | | | |
|---|---|---|---|---|---|---|---|---|---|---|
| | | N₂ flow | Bubbler | vaporised | | Reactor | CO₂ flow | Carbamate | | Powder |
| Example | Amine | rate | temperature | polyamine | | temperature | rate | produced | | grain size |
| 3 | 1,3-phenylenediamine | 100 slm | 100° C. | 0.59 | g/min | 100° C. | 0.2 slm | 50 | g/h | <2 μm |
| 4 | 1,3-diaminopropane | 100 slm | 60° C. | 23.15 | g/min | 60° C. | 7.0 slm | 1.91 | kg/h | <4 μm |
| 5 | 1,4-diaminocyclohexane | 100 slm | 100° C. | 77.45 | g/min | 100° C. | 23.0 slm | 6.43 | kg/h | <5 μm |
| 6 | 3-aminomethyl-3,5,5- | 100 slm | 100° C. | 1.92 | g/min | 100° C. | 0.4 slm | 144 | g/h | <2 μm |
| | trimethylcyclohexyl- | | | | | | | | | |
| | amine | | | | | | | | | |
| 7 | 1,12-diaminododecane | 100 slm | 140° C. | 5.8 | g/h | 140° C. | 0.1 slm | 7 | g/h | <2 μm |
| 8 | 4,4′-methylenebis- | 100 slm | 140° C. | 352 | g/h | 140° C. | 2.0 slm | 0.427 | kg/h | <4 μm |
| | (cyclohexylamine) | | | | | | | | | |

As may be seen from the data shown in Table 1, the process according to the invention, apart from being operationally straightforward, makes it possible to obtain finished carbamate particles which are in each case smaller than 5 μm. Of course, this value of the carbamate particle size obtained may be adjusted both by acting on the residence time of the reactants within the reactor and by making use of nozzles with multiple injection points, as is known from aerosol reactor design methods.

Although the invention has been described with reference to primary polyamines and with separate apparatuses for the two characteristic steps of the process, modifications and additions may be made by the person skilled in organic chemistry with the aim of making the described and claimed process suitable for application to secondary and/or mixed polyamines, without departing from the scope of the appended claims.

1-20. (canceled)

21. A process for the preparation of carbamates of primary and/or secondary polyamines, which process comprises the following steps:
a) vaporising a primary and/or secondary polyamine,
b) reacting said vaporised primary and/or secondary polyamine with gaseous carbon dioxide.

22. The process according to claim 21, wherein, if the primary and/or secondary polyamine is solid at ambient temperature, it is melted before step a).

23. The process according to claim 21, wherein the polyamine is selected from the group consisting of aliphatic, cycloaliphatic or aromatic diamines.

24. The process according to claim 23, wherein aliphatic, cycloaliphatic or aromatic diamines have a number of carbon atoms in the range from 2 to 14.

25. The process according to claim 24, wherein the polyamine is selected from the group consisting of 1,2-diaminopropane, 1,6-diaminohexane, 1,2-diaminoethane, 1,4-diaminobutane, 1,5-diaminopentane, 1,10-diaminodecane, 1,12-diaminododecane, 1,4-diaminocyclohexane, 4,4′-methylenebis(cyclohexylamine), 1,4-phenylenediamine, 1,3-phenylenediamine, 1,4-diaminocyclohexane and 3-aminomethyl-3,5,5-trimethylcyclohexylamine.

26. The process according to claim 25, wherein the polyamine is selected from the group consisting of hexamethylenediamine [1,6-diaminohexane], 1,12-diaminododecane, 4,4′-methylenebis(cyclohexylamine).

27. The process according to claim 21, wherein the polyamine vaporisation step a) proceeds in the presence of a carrier gas.

28. The process according to claim 21, wherein the reaction step b) proceeds in the presence of a propellant gas.

29. The process according to claim 27, wherein the carrier gas is an inert gas.

30. The process according to claim 28 wherein the propellant gas is an inert gas.

31. The process according to claim 29, wherein the inert gas is selected from the group consisting of nitrogen, air and nitrogen-enriched air.

32. The process according to claim 30, wherein the inert gas is selected from the group consisting of nitrogen, air and nitrogen-enriched air.

33. The process according to claim 21, wherein the polyamine vaporisation step a) is performed by bubbling said carrier gas into the polyamine which is maintained in the liquid phase.

34. The process according to claim 21, wherein the vaporisation step a) is performed in a flash chamber.

35. The process according to claim 21, wherein step b) is performed under turbulent flow conditions.

36. The process according to claim 21, wherein step b) is performed under laminar fluid dynamic conditions.

37. The process according to claim 21, wherein, in step b), the carbon dioxide is present in the reaction mixture in an amount at least stoichiometrically equivalent to the amount of polyamine.

38. The process according to claims 21, wherein, in step b), the carbon dioxide is in stoichiometric excess with respect to the polyamine.

39. The process according to claim 27 wherein, when the carrier gas is nitrogen, said carrier gas, on leaving step b), is purified and recirculated upstream of the vaporisation step a).

40. The process according to claim 28, wherein the propellant gas is supplied independently of the polyamine and the carbon dioxide.

41. The process according to claim 21, wherein the reaction step b) proceeds in a continuously operated synthesis reactor.

42. The process according to claim 21, wherein the reaction step b) proceeds in a semi-continuously operated synthesis reactor.
